(19) United States
(12) Patent Application Publication
(10) Pub. No.: US 2003/0116165 A1
(43) Pub. Date: Jun. 26, 2003

(54) Earpiece

(76) Inventor: Hsin-Fu Huang, Taoyuan Hsien (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/023,658

(22) Filed: Dec. 21, 2001

(51) Int. Cl. A61F 11/00

(52) U.S. Cl. 128/864

(57) ABSTRACT

An earpiece having a hollow cylindrical earpiece body and a diaphragm suspended at one end of the hollow cylindrical earpiece body for protecting the ear against outside water and for enabling the ear to hear a certain volume of sound.

### BACKGROUND OF THE INVENTION

1. Field of the Invention

The present invention relates to earpieces and, more particularly, to an earpiece effectively prohibits water from passing through but allows a certain volume of sound waves to pass.

2. Description of the Related Art

A conventional earpiece 6, as shown in FIG. 12, is a solid, cylindrical cork. When in use, the earpiece 6 is plugged into the external auditory canal 71 of the ear 7 to isolate outside noises. After installation of the earpiece 6 in the ear 7, the user cannot hear the broadcasting or calling from another person during an emergency. Further, an user may use the earpiece 6 to seal the ear against water when swimming. Because the user who worn the earpiece in each ear cannot hear the warning broadcasting or calling from another person during a dangerous situation, a tragic accident may happen. In addition to the aforesaid problem, the installation of the earpiece 6 in the ear may cause the user to lose the balance and to suffer headache.

### SUMMARY OF THE INVENTION

The present invention has been accomplished under the circumstances in view. It is the main object of the present invention to provide an earpiece, which eliminates the aforesaid problems. According to one embodiment of the present invention, the n earpiece has a hollow cylindrical earpiece body and a diaphragm suspended at one end of the hollow cylindrical earpiece body for protecting the ear against outside water and for enabling the ear to hear a certain volume of sound. According to an alternate form of the present invention, the earpiece comprises a hollow cylindrical earpiece body and an end cap fastened to one end of the hollow cylindrical earpiece body. The end cap has a thin layer of base that protects the ear against outside water and allows the ear to hear a certain volume of sound when the user is swimming.

### BRIEF DESCRIPTION OF THE DRAWINGS

FIG. 1 is a sectional elevation of an earpiece constructed according to a first embodiment of the present invention.

FIG. 2 is a plain view of the earpiece according to the first embodiment of the present invention.

FIG. 3 is similar to FIG. 2 but showing the diaphragm made relatively thicker.

FIG. 3A is similar to FIG. 2 but showing the uniform wall thickness of the diaphragm and the peripheral wall of the hollow cylindrical earpiece body.

FIG. 4 is sectional elevation of an alternate form of the earpiece constructed according to the first embodiment of the present invention.

FIG. 5 is an exploded view in section of an earpiece constructed according to a second embodiment of the present invention.

FIG. 6 is an exploded view in section of an earpiece constructed according to a third embodiment of the present invention.

FIG. 7 is a sectional view of an earpiece constructed according to a fourth embodiment of the present invention.

FIG. 8 is an elevational view of an earpiece constructed according to a fifth embodiment of the present invention.

FIG. 9 is a sectional view of the earpiece according to the fifth embodiment of the present invention.

FIG. 10 is an elevational view of an earpiece constructed according to a sixth embodiment of the present invention.

FIG. 11 is a sectional view of the earpiece according to the sixth embodiment of the present invention.

FIG. 12 illustrates the application of an earpiece according to the prior art.

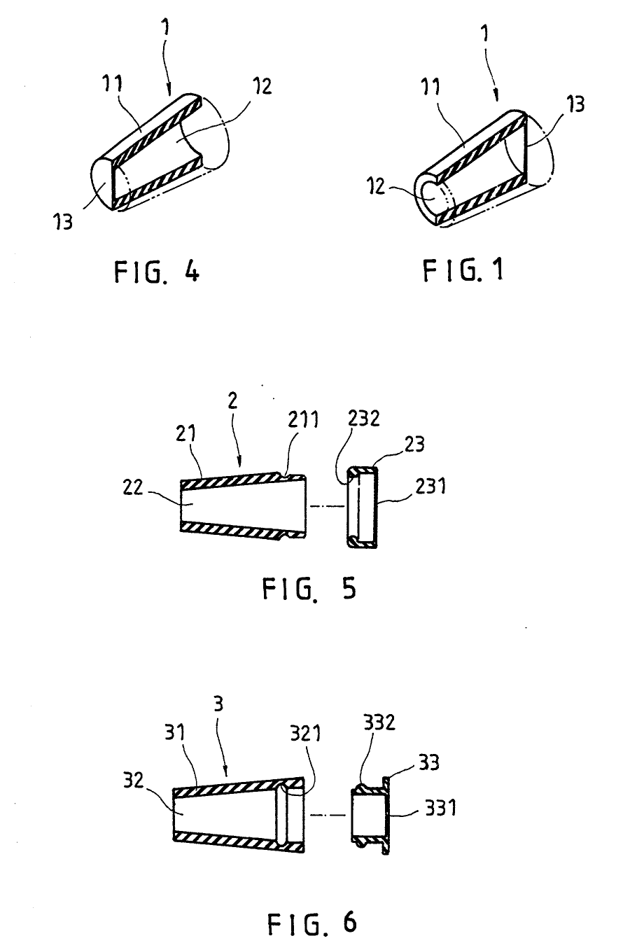

### DETAILED DESCRIPTION OF THE PREFERRED EMBODIMENT

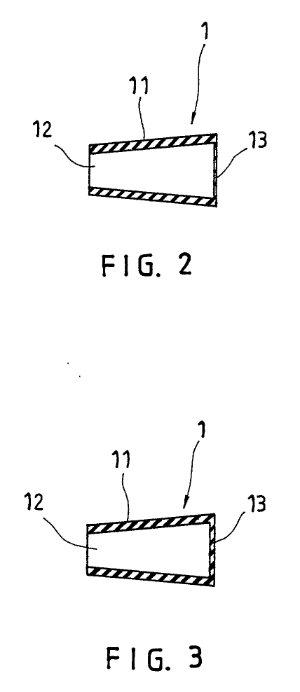

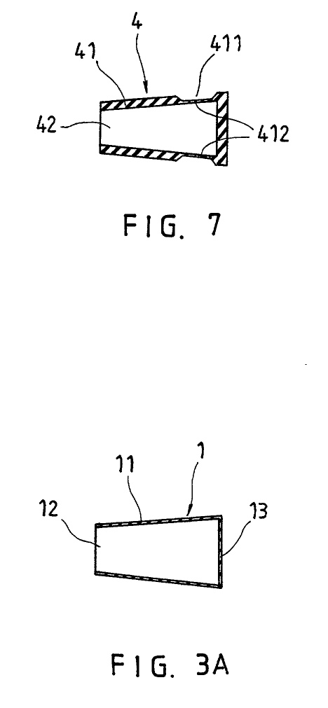

Referring to FIGS. from 1 through 4, an earpiece 1 is shown comprising a hollow cylindrical body 11 shaped line a truncated cone and defining a chamber 12, and a diaphragm 13 suspended at one end of the chamber 12. The diaphragm 13 prohibits water from passing through the chamber 12 to the inside of the user's ear but allowing the ear to hear certain volume of sound. Therefore, the user bearing the earpiece 1 can still hear the output sound of a broadcasting unit. The thickness of the diaphragm 13 may be determined subject to actual requirement (see FIGS. 2 and 3). FIG. 3A shows that the diaphragm 13 and the peripheral wall of the body 11 have same wall thickness. Further, the diaphragm 13 may be provided at one end, namely, the big end of the hollow cylindrical body 11 (see FIGS. from 1 through 3), or the other end, namely, the small end of the hollow cylindrical body 11 (see FIG. 4).

FIG. 5 shows an ear piece constructed according to a second embodiment of the present invention. According to this embodiment, the earpiece 2 comprises a hollow cylindrical body 21, and an end cap 23 fastened to one end of the body 21. The body 21 has an outside annular coupling groove 211 around the periphery thereof at one end, and an open chamber 22 axially extended through the two distal ends thereof. The end cap 23 comprises a thin layer of base 231 and an annular coupling flange 232 extended from the inside wall of the cap body thereof for coupling to the coupling groove 211 of the body 21. After connection of the coupling flange 232 to the coupling groove 211, the end cap 23 and the body 21 are positively secured together in a flush manner. Further, the thickness of the thin layer of base 231 of the end cap 23 is determined subject to actual requirement.

FIG. 6 shows an ear piece constructed according to a third embodiment of the present invention. According to this embodiment, the earpiece 3 comprises a hollow cylindrical body 31, and an end cap 33 fastened to one end of the body 31. The body 31 comprises an inside annular coupling groove 321 around the inside wall thereof near one end and an open chamber 32 axially extended through the two distal ends thereof. The end cap 33 comprises a thin layer of base 331 and an outside annular coupling flange 332 extended around the periphery of the cap body thereof for coupling to the inside annular coupling groove 321. After insertion of the end cap 33 into one end of the body 3, the outside annular coupling flange 332 is forced into engagement with the inside annular coupling groove 321, and the thin layer of base 331 is stopped at one end of the body 3.

FIG. 7 shows an earpiece constructed according to a fourth embodiment of the present invention. According to this embodiment, the earpiece 4 comprises a hollow cylindrical body 41 an open chamber 42. The hollow cylindrical body 41 has one end opened, the other end closed, and an outside annular groove 411 disposed around the periphery thereof near the close end, and a thin wall portion 412 surrounded by the outside annular groove 411. The thin wall portion 412 enables certain volume of sound waves to pass.

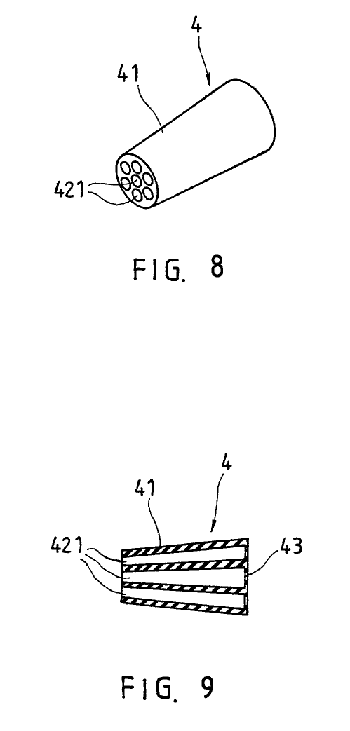

FIGS. 8 and 9 show an earpiece constructed according to a fifth embodiment of the present invention. According to this embodiment, the earpiece 4 comprises a tapered, cylindrical body 41, a plurality of axial holes 421 axially extended to one end, and a diaphragm 43 disposed at the other end. The diaphragm 43 prohibits water and high noises from passing through the axial holes 421 to the inside of the ear in which the earpiece 4 is installed, but allowing a certain amount of sound waves to pass.

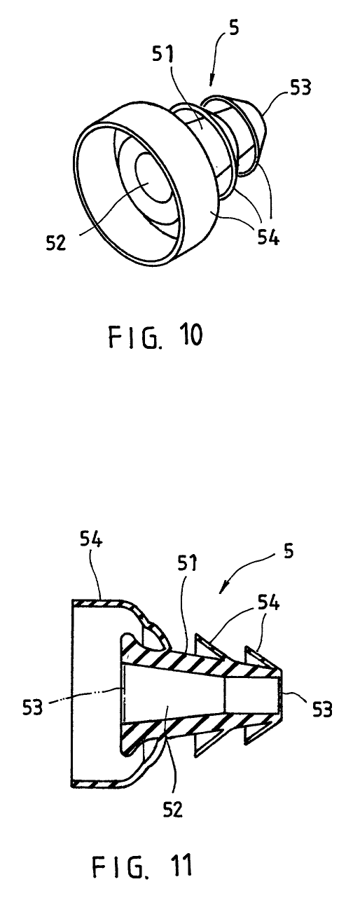

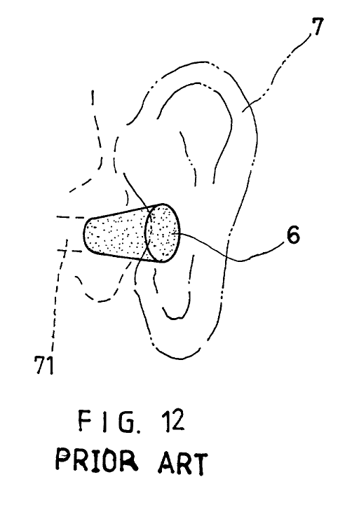

FIGS. 10 and 11 show an earpiece constructed according to a six embodiment of the present invention. According to this embodiment, the earpiece 5 comprises a hollow cylindrical body 51, two diaphragms 53 respectively disposed at the ends of the hollow cylindrical body 51, an enclosed chamber 52 defined in the hollow body 51 between the diaphragms 53, and flexible conical skirts 54 extended around the periphery of the hollow cylindrical body 51 and axially spaced from one another. The flexible conical skirts 54 have different diameters arranged in proper order.

Although particular embodiments of the invention have been described in detail for purposes of illustration, various modifications and enhancements may be made without departing from the spirit and scope of the invention. Accordingly, the invention is not to be limited except as by the appended claims.

What the invention claimed is:

1. An earpiece comprising a hollow cylindrical earpiece body and a diaphragm suspended at one end of said hollow cylindrical earpiece body.

2. The earpiece as claimed in claim 1, wherein said hollow cylindrical earpiece body has a peripheral wall of thickness equal to said diaphragm.

3. The earpiece as claimed in claim 1, wherein said hollow cylindrical earpiece body is shaped like a truncated cone.

4. An earpiece comprising a hollow cylindrical earpiece body, said hollow cylindrical earpiece having an open chamber axially extended through two distal ends thereof, and an end cap fastened to one end of said hollow cylindrical earpiece.

5. The earpiece as claimed in claim 4, wherein said hollow cylindrical earpiece body has an outside annular coupling groove disposed around the periphery of one end thereof, and said end cap has an inside annular coupling flange coupled to the outside annular coupling groove of said hollow cylindrical earpiece body.

6. The earpiece as claimed in claim 4, wherein said hollow cylindrical earpiece body has an inside annular coupling groove extended around an inside wall thereof near one end; said end cap has a cap body plugged into one end of said hollow cylindrical earpiece body, and an outside annular coupling flange disposed around said cap body and forced into engagement with the inside annular coupling groove of said hollow cylindrical earpiece body.

7. An earpiece comprising a hollow cylindrical earpiece body and an open chamber defined in said hollow cylindrical earpiece body, said hollow cylindrical body a first end opened, a second end closed, an outside annular groove disposed around the periphery thereof near the close end, and a thin wall portion surrounded by said outside annular groove.

8. The earpiece as claimed in claim 7, wherein said hollow cylindrical earpiece body is shaped like a truncated cone.

9. An earpiece comprising a tapered earpiece body, said tapered earpiece body having a first end and a second end, a plurality of axial holes axially disposed in said tapered earpiece body and extended through the first end of said tapered earpiece body, and a diaphragm disposed at the second end of said tapered earpiece body.

10. An earpiece comprising a hollow cylindrical earpiece body, two diaphragms respectively disposed at two distal ends of said hollow cylindrical earpiece body, an enclosed chamber defined in said hollow body between said diaphragms, and flexible conical skirts extended around the periphery of said hollow cylindrical earpiece body and axially spaced from one another.

11. The earpiece as claimed in claim 10, wherein said flexible conical skirts have different diameters arranged in proper order.
